(19) United States
(12) Patent Application Publication
(10) Pub. No.: US 2025/0043362 A1
(43) Pub. Date: Feb. 6, 2025

(54) SELECTIVE DIGITAL MULTIPLEXING

(71) Applicant: Bio-Rad Laboratories, Inc., Hercules, CA (US)

(72) Inventors: George Karlin-Neumann, Palo Alto, CA (US); Ronald Lebofsky, Kensington, CA (US); Mona Shahbazian, Hercules, CA (US); Mina Rostamza, Hercules, CA (US)

(21) Appl. No.: 18/793,760

(22) Filed: Aug. 3, 2024

Related U.S. Application Data
(60) Provisional application No. 63/530,900, filed on Aug. 4, 2023.

(51) Int. Cl. C12Q 1/6886; C12Q 1/6806; C12Q 1/6809; C12Q 1/686; G01N 21/64

(52) U.S. Cl. CPC C12Q 1/6886; C12Q 1/6806; C12Q 1/6809; C12Q 1/686; G01N 21/6486; C12Q 2600/16

(57) ABSTRACT

The invention provides methods for the detection of molecular targets by digital PCR (dPCR) with a blocker for non-target molecules. Each target is provided with a unique mixture of probes. The blocker binds to a non-target molecule and blocks it from contributing to fluorescence from partitions. Two or more colors of fluorescence intensity are read, and two colors of fluorescence intensity are plotted as a 2D plot. In the plot, different targets contribute well-resolved clusters. Each cluster in the plot essentially lies a long its own radius allowing for radial multiplexing. The use of a blocker selects against the detection of non-target molecules and improves the resolution of radial multiplexing, allowing multiple targets to be detected.

### TECHNICAL FIELD

The disclosure relates to the detection of molecular targets.

### BACKGROUND

Numerous clinical and research methods involve the detection of specific nucleic acids. For example, cancer is typically associated with certain mutations in tumor DNA and the ability to detect those mutations can be informative of the presence or progress of the cancer or the success of a treatment. In theory, after a patient is treated to remove a tumor, the success of the treatment can be evaluated by performing an assay to detect tumor DNA. It is understood that tumor cells and cell-free tumor DNA can be found circulating in blood. Detecting such circulating tumor nucleic acid in a laboratory assay would be a valuable diagnostic tool.

Unfortunately, circulating tumor DNA is only a minor fraction of a blood draw, which makes it a challenge to detect. Such a sample also includes abundant DNA from non-tumor cells. Additionally, such tumor DNA may have a diverse variety of different genetic mutations, each of which may be clinically significant. Similar challenges arise in pathogen detection, agriculture, and other fields of endeavor. For example, there is increasing interest in assaying wastewater to monitor patterns of viral spread in communities. However, in such a sample, the target of interest may be present only in minor fractions and may also have genetic diversity among those viral nucleic acids that are present. For example, a wastewater system that serves a large metropolitan region may have trace amounts of viral nucleic acid that are variously derived from different variants of a virus that is spreading through the region.

Some approaches to detecting such nucleic acids of interest have involved capture and amplification of those targets by, for example, polymerase chain reaction (PCR). However, PCR based assays may not be fully satisfactory for certain samples, particularly where the targets of interest are only present in small quantities, especially if among abundant quantities of similar, but less-informative molecules, or where there is diversity among the targets of interest.

### SUMMARY

The invention provides methods for the detection of molecular targets such as nucleic acids using highly multiplexed digital PCR (dPCR) with blocker molecules that inhibit the detection of non-target molecules in a sample. For the multiplex dPCR of the invention, a sample is divided into a large number (tens of thousands) of aqueous partitions and a PCR reaction is conducted in each partition with primers and probes that generate amplicons and give a detectable signal when a target of interest is present in a partition. Each partition can include probes specific to several different targets (e.g., three, four, five, six, or seven, or more) and methods of the invention allow the presence of each target to be read even when using fewer detection channels than there are targets. Certain embodiments use fluorescent hydrolysis probes that are read in two or more optical channels at a time. Each target is provided with a unique mixture of probes that will fluoresce in the two or more channels. Two or more colors of fluorescence intensity are read (using optical detectors such as photodiodes) and the fluorescence intensity may be stored as a 2D plot with one axis for each color. Each target that is present in a partition will appear as one distinct cluster of points on the plot. By careful probe design according to methods of the disclosure, individual clusters are well-resolved, and software can be used to model a Poisson distribution of targets into partitions and report what quantities of the targets in the original sample generate the observed clusters. The readout is thus multiplexed by virtue of the independent detection of multiple targets from one, two (or more) color detection operations.

Highly multiplexed detection of rare targets is promoted by the use and inclusion of a blocker. The blocker is a molecule or molecular species that binds to or suppresses at least one non-target molecule from contributing to fluorescence from the partitions. For example, the blocker can be an oligonucleotide that is perfectly complementary to a nucleic acid that is not the intended target of the dPCR assay. The blocker will bind to the non-target and to amplicon copies thereof. The blocker may inhibit or displace any fluorescent probe from binding to those molecules, or the blocker may impede one of the primers from binding to those molecules thus inhibiting amplification of the non-target and removing the non-target molecule from the dPCR readout. Due to that inhibition, the blocker removes the non-target molecule from the dPCR readout. The multiplex probe combination and blocker work particularly well together to prevent wild-type sequences from being read in a dPCR assay for rare mutations or variants. With methods of the invention, a plurality of cancer-associated mutations can be reliably detected in a multiplex digital PCR assay while two wild-type alleles are blocked from being read.

When the variants to be detected are read in two or more color channels, the resulting plot has well-resolved clusters. Each cluster in the plot lies along its own radius from the cluster for double negative partitions and detecting those independent clusters may be referred to as radial multiplexing. The use of a blocker selects against the detection of non-target molecules and improves the resolution of radial multiplexing, allowing five, six, seven or more independent targets (e.g., variants) to be detected in the results. The selective digital multiplexing provides for the reliable quantification of multiple targets in a sample.

In certain aspects, the invention provides target detection methods. Method of the invention include partitioning, into a plurality of aqueous partitions, a sample that includes, or potentially includes, or is suspected of including, or is being tested for the presence or absence of, one or more of at least four different nucleic acid variants. The portioning is performed such that the partitions include amplification reagents, variant-specific probes, and a blocker that inhibits fluorescence from amplification of a fourth one of the variants. Each of the variant-specific probes (e.g., fluorescent hydrolysis probes) is specific to one of three of the variants and produces fluorescence of a first color or a second color. Notably, at least some of the targets are targeted by a mixture of probes with two or more different colors of fluorescent reporter. To illustrate, a first target may be targeted using only probes with a carboxyfluorescein reporter (FAM), a third target may be targeted using only probes with a hexachlorofluorescein reporter (HEX), and a second target may be targeted with a mixture or probes, some with FAM, some with HEX. More complex multiplexing schemes using more other probe mixtures are described herein.

The partitions are subject to conditions that promote amplification (e.g., thermocycling) and the methods include reporting the presence or absence of the three of the variants in the sample based on amounts of the first color and the second color detected from the partitions. The fourth one of the variants may be a wild-type and the three of the variants are mutant version of the wild-type and the blocker comprises an oligonucleotide with no fluorescent label. The methods provide a digital PCR (dPCR) assay with a two-color readout in which more than two targets can each be independently detected in a sample. Notably, where the targets of-interest are variants, or mutants, of some wild-type gene (such as estrogen receptor 1, for example), the blocker can suppress amplification-generated fluorescence of the wild-type, allowing the dPCR readout to have greater discriminating power for the variants, which may each be a very minor fraction of original sample, relative to the wild type.

In support of readouts such as dPCR, the methods involve portioning the sample and reagents into partitions. The partitions may be droplets, wells in a plate, or other fluid portioning structure. Methods preferably include diluting the sample so that each partitions receives a limited number of target molecules, such as zero, one, two, sometimes three, and a very small number of four or more. The dilution can be calculated so that a majority of partitions receive a target number (e.g., zero or one) of targets. Each target molecule will serve as a template for generation of amplicons in the presence of fluorescence probes. Preferred probes include an oligonucleotide backbone that anneals in a sequence specific manner to a target of that probe, plus a fluorophore and a quencher. Methods may include thermocycling the droplets within a reaction tube or well of a plate. During amplification with polymerase, exonuclease activity of the fluorophore digests the oligonucleotide backbone of any bound probe, separating the fluorophore from the quencher, allowing the fluorophore to fluoresce during a readout step. To read fluorescence, methods may include flowing the droplets (e.g., one-at-a-time) past a detector (and optionally a light source for excitation of fluorophores).

Methods may be used for multiplex detection of multiple targets with two-channel readout at a time. For example, five targets may be read in two color channels. In some embodiments, four or six or more color channels are used, two at a time during readout. For example, the method may include reading the sample for at least seven variants using at least six colors, wherein the detecting step reads two of the six colors, in two channels, at a time. Alternatively, other combinations of channels and read points may be used (e.g., 2 colors at 1 read point, 2 colors at 2 read points, 2 colors at 3 read points, 3 colors at 1 read point, 3 colors at 2 read points or 4 colors at 1 read point). The six colors may be provided by any suitable fluorophores or fluorescent dyes. Some embodiments use six fluorescent reporters that include carboxyfluorescein (FAM), hexachlorofluorescein (HEX), cyanine5 (CY5), cyanine5.5 (CY5.5), 5-carboxy-X-rhodamine (5-ROX), and a fluorescent oligonucleotide dye with 594 nm adsorption (ATTO590).

Methods may be used for radial multiplexing, which can be implemented by reading two or more colors from each of a plurality of partitions, plotting intensity of the two colors on a 2D plot with axes for intensity of each color, and identifying clusters of points on the plot. Each cluster will typically be found lying along distinct radius extending from a cluster corresponding to double-negative (no significant fluorescence of either color) partitions. In radial multiplexing, clusters corresponding to distinct targets in the sample are distinguishable according to their different radial directions from the double negative cluster. Clusters can further be distinguished based on radial distance. Thus, five or more targets can be distinguished by providing mixtures of probes with varying amounts of the two fluorescent reporters interrogated in the two channels. Other methods of multiplexing such as fluorescence intensity multiplexing are within the scope of the disclosure. For radial multiplexing, the reporting step may include plotting the amounts of the first color and the second color detected from the partitions as points on a graph and identifying clusters of the points on the graph corresponding to the presence of any of the three variants. The quantities of the respective targets in the sample may be determined by a Poisson model of templates into the partitions that would generate the observed pattern of clusters.

The amplification step may include thermocycling, using reagents that include PCR primers and dNTPs. Preferably, the variant-specific probes include fluorescent hydrolysis probes that each anneal to a sequence of one of three of the variants. The blocker may include an oligonucleotide that anneals to a wild-type version of the variants and inhibits amplification of wild type sequences. In some cases, the blocker may include an oligonucleotide that anneals to a wild-type version of the variants and prevents fluorescent probes from binding to the wild-type version or amplicons thereof. The blocker may include other features (e.g., one or more locked-nucleic acids in the oligonucleotide) or other molecular strategies. For example, in some embodiments, the blocker is a binding protein (such as an RNA-guided binding protein) that binds to the fourth one of the variants and prevents the amplification.

The reporting step may include detecting colors from the partitions, two or more color channels at a time, in three detection operations for six colors. The reporting step may include reporting the presence or absence of at least seven mutations of a gene being present in the sample. In some embodiments, the method is used to detect mutant versions of, or variants of, a gene. The gene may be estrogen receptor 1 (ESR1) and one or more of the seven mutations may be selected from the group consisting of c1138G>C, c.1387T>C, c1607T>G, c.1609T>A, c1610A>C, c1610A>G, and c.1613A>G in the coding sequence of the ESR1 gene.

In certain embodiments, the variants are present in tumor DNA in the sample, and the method (i) includes isolating circulating tumor DNA (ctDNA) from the sample, or (ii) isolating circulating tumor cells (CTCs) from the sample and purifying DNA from the CTCs.

Preferably, each of the three of the variants is targeted with a respective probe or probe combination that includes a sequence specific to that variant and zero or a characteristic amount of the first color or the second color. In some embodiments, a first probe combination specific to a first variant include only probes with a first sequence and the first color, a second probe combination specific to a second variant includes a subset of probes with a second sequence and the first color and a subset of probes with a second sequence and the second color, and a third probe combination specific to a third variant includes only probes with a third sequence and the second color. Optionally, amounts of the first color and the second color detected from the partitions are plotted against respective first and second axes on a plot, wherein the three of the variants each form a respective distinct cluster on the plot, wherein the distinct clusters can be separated by radii extending from one point.

Other aspects of the invention provide a multiplex digital PCR method that includes dividing a sample that includes up to four distinct nucleic acid targets into a plurality of aqueous partitions that also include detectably labeled probes for three of the targets and a blocker that inhibits, during amplification conditions, production of a detectable signal from a fourth one of the targets. The method includes exposing the partitions to the amplification conditions and detecting the presence or absence of each of the three of the targets in the sample by reading signal in two optical channels. The detecting step may be done by plotting signal intensity from each partition on a plot with an axis for each channel, such that each of the three targets, if present in the sample, forms a distinct cluster on the plot. For those targets that are present in the sample, the plot may include clusters that are located along their own radii extending from a point (the double-negative point) on the plot.

In some embodiments, when more than three targets are present, the method may detect the presence or absence of each of the targets in the sample by reading signal in more than two optical channels, or reading signal from the sample more than once using different channels for each read.

In some embodiments, a first one of the three targets is detected only using probes that include a first oligonucleotide sequence linked to a label that produces a first color, a second one of the three targets is detected using a mixture of probes that include a second oligonucleotide sequence linked to a label that produces the first color and the second nucleotide sequence linked to a label that produces a second color, and a third one of the three targets is detected using only probes that include a third oligonucleotide sequence linked to a label that produces the second color. The blocker may be an oligonucleotide that binds to the fourth one of the targets, or copies thereof, and inhibits binding of any of the detectably labeled probes.

In highly multiplex embodiments, the sample may include up to eight targets and the method may include detecting the presence or absence of least seven of the eight targets by three two-color reading operations over a total of six colors. In some embodiments, the method may include detecting the presence or absence of least seven of the eight targets by one or more reading operation, each reading two or more colors. The four distinct nucleic acid targets may include homologous genetic sequences. The targets may be variants of a wild-type gene, where the variants may be of, e.g., clinical significance, and the blocker is used to suppress the wild-type out of digital PCR readouts. For example, the blocker may inhibit detection of a wild-type sequence of a gene where each of the targets include a mutated portion of the gene (i.e., that includes a mutation, relative to the wild-type sequence). Exemplary genes may include BRAF, EGFR, KRAS, NRAS, PIK3CA, or ESR1. In certain embodiments, the sample includes tumor DNA from a subject, the blocker suppresses detection of wild-type sequences from non-tumor DNA from the subject, and detection of the three targets (presence or absence of each) shows the presence or grade of a tumor in the subject.

The blocker may include a protein that binds specifically to the fourth one of the targets or amplicons thereof. The blocker may be, for example, an RNA-guided binding protein.

The method may include estimating quantities of each of the three of the targets in the sample by modelling, using a computer system, a Poisson distribution of the three of the targets that would give the reading of the signal in the two optical channels.

The four distinct nucleic acid targets may include (i) a first mutation of a wild-type sequence, (ii) a second mutation of the wild-type sequence, (iii) a third mutation of the wild-type sequence, and (iv) the wild-type sequence. The detectably-labeled probes may include: a first probe that includes a first oligonucleotide that anneals to the first mutation with a fluorophore of a first color linked to the first oligonucleotide; a second probe that includes second oligonucleotide that anneals to the second mutation with the fluorophore of the first color linked to the second oligonucleotide; a third probe that includes the second oligonucleotide that anneals to the second mutation with a fluorophore of a second color linked to the second oligonucleotide; and a fourth probe that includes a third oligonucleotide that anneals to the third mutation with a fluorophore of a third color linked to the third oligonucleotide.

In certain embodiments, the method involves detecting the presence or absence of each of five distinct nucleic acid targets in two optical channels, wherein the five distinct nucleic acid targets include target one, target two, target three, target four, and target five, and wherein the detectably labeled probes include fluorescent hydrolysis probes with five respective oligonucleotides, wherein: (i) all probes for target one have a first fluorophore, (ii) a majority of probes for target two have the first fluorophore and a remainder of the probes for target two have a second fluorophore, (iii) probes for target three have approximately equal amounts of the first and the second fluorophore, (iv) a minority of probes for target four have the first fluorophore and a remainder of the probes for target four have a second fluorophore, and (v) all probes for target five have the second fluorophore. In some embodiments, the five distinct nucleic acid targets are variants of a wild-type gene sequence, and the blocker inhibits the fluorescent hydrolysis probes from annealing to the wild-type gene sequence or amplicons thereof.

Embodiments of the invention provide for multiplexing five or more targets in a two-channel dPCR reading, e.g., via radial multiplexing. Multiplex reading of at least five targets in two channels may include detecting the presence or absence of each of five distinct nucleic acid targets in the two optical channels. The five distinct nucleic acid targets may include target one, target two, target three, target four, and target five. Preferably, the detectably labeled probes include fluorescent hydrolysis probes with five respective oligonucleotides, each linked to one of a first and second fluorophore such that no two of the five distinct nucleic acid targets are probed with matching quantities of the first and second fluorophore.

While described as being read in two optical channels, the scope of the invention is not limited to the number of channels read at a time. For example, in any of the embodiments described herein, one, two, three, four, or more, channels or colors may be read at a single time point. Further, the scope of the invention also includes multiple sample reads across different color channels. For example, one four-channel read may be performed, or two, two-channel reads may be performed.

### BRIEF DESCRIPTION OF THE DRAWINGS

FIG. 1 diagrams steps of a method.

FIG. 2 illustrates the multiplex detection of four variants.

FIG. 3 shows a plot obtained when using a blocker.

FIG. 4 illustrates reagents including probes and blocker.

FIG. 5 shows a 2D plot of FAM and HEX from partitions.

FIG. 6 shows a 2D plot of CY5 and CY5.5.

FIG. 7 shows a 2D plot for ROX and ATTO590.

FIG. 8 shows reading of WT ESR1 gene.

FIG. 9 shows a second reading of a WT ESR1 gene.

FIG. 10 is a final plot generated by the instrument in the ESR1 WT assay.

FIG. 11 illustrates results of a fluorescent intensity multiplexing assay.

### DETAILED DESCRIPTION

The invention provides methods for detecting targets of interest and in particular provides for the use of digital PCR based detection for the multiplex detection of multiple genetic targets in a single readout operation. Preferred embodiments are useful for the detection of three or more distinct genetic targets in a readout operation that employs only two color channels at a time. Additionally, methods of the invention make use of a blocker that suppresses or inhibits the ability of one nucleic acid sequence to generate any signal in a digital PCR assay. The blocker is particularly useful where several sequences are related and those sequences of interest are certain mutations, or variants, of a predominant wild-type sequence. When those variants are of clinical interest, a digital PCR assay of the disclosure may be performed using fluorescent reporters that give distinct signals for each variant of-interest while a blocker minimizes any signal from the wild-type sequence.

For example, the digital PCR assay may involve amplification of target nucleic acids from a sample in aqueous partitions with a primer pair. The sample may include some number of variants of interest as well as an abundant number of copies of the wild-type sequence. In such a case, those templates are genetic homologs. Those templates (the wild-type plus the variants) are amplified by the primer pair. The partitions include detectably labeled probes for the variants of-interest. Because of the homology among those templates, some variant-specific probes may anneal to the wild-type template, or amplicons thereof. Were such binding to happen, the wild-type template would generate a signal that would be detected in the final readout. The invention uses a blocker to inhibit signal from the wild-type template. For example, the blocker may be an oligonucleotide that binds specifically to the wild-type template. To maximize its effect as a blocker, the blocker may include a wild-type specific base at one end, may include one or more locked nucleic acids, may be designed against a GC-rich stretch of the wild-type template, or other such features or combinations thereof. The blocker may include a fully wild type sequence. The blocker may include a blocking moiety at one end to prevent extension by a DNA polymerase. For example, the blocker may include a blocking moiety at the 3′ end to prevent extension by DNA polymerase. Such blocking moieties may include modifications such as minor groove binders, inverted DNA nucleotides, 3-carbon spacers, modified or unmodified nucleotides (e.g., 1, 2, 3, 4, or more nucleotides) that do not pair with the template, or other moieties known in the art. In some embodiments, during PCR with, e.g., fluorescent hydrolysis probes, the blocker anneals to wild-type sequences and inhibits amplification and non-specific binding by variant-specific probes. In other embodiments, during PCR with, e.g., fluorescent hydrolysis probes, the blocker may anneal to template and/or amplicons thereof and cannot be displaced by variant-specific probes. By virtue of the blocker, each aqueous partition generates substantial fluorescence only when one of the probe-targeted variants is in that partition.

By virtue of its inhibition of detectable signal from a dominant species, the blocker facilitates the successful multiplex detection of the various minor species in the sample. That is, multiple (e.g., 3, 4, 5, or more) variants can be detected in a single two-channel readout and those variants will form well-defined and distinguishable clusters in part because the dominant wild-type has been suppressed. While stated here in terms of mutant variants and a wild-type sequence, methods of the invention promote successful multiplex detection in numerous contexts and biological situations, particularly where there are multiple minor (in quantity) targets of interest in a sample that includes a predominant target. For example, multiplex detection according to methods of the invention may use a blocker to suppress healthy, non-tumor sequences when assaying for tumor mutations. Methods may use a blocker to suppress signal from maternal DNA when assaying for circulating fetal nucleic acids. Methods of the invention may be used for the multiplexed detection of pathogenic strains of a bacteria within a sample dominated by a benign strain. Methods of the invention may be used to detect multiple variants of a virus (e.g., in wastewater or sewage) while blocking detection of a variant for which the public has received an effective vaccine.

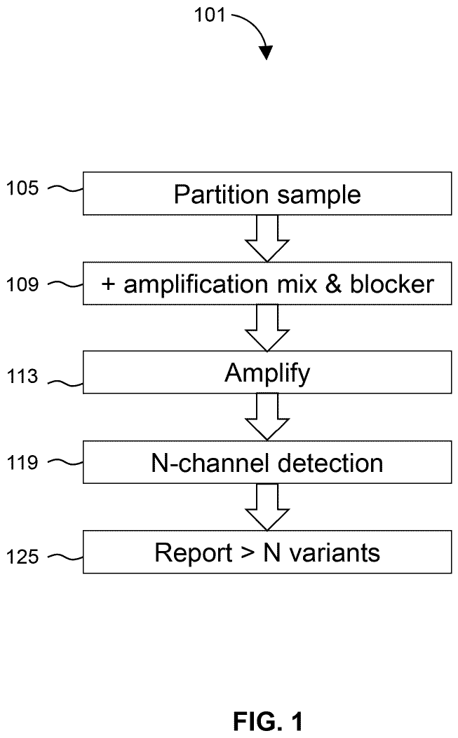

FIG. 1 diagrams steps of a method 101. The method 101 includes partitioning 105, into a plurality of aqueous partitions, a sample that includes, or potentially includes, or is suspected of including, or is being tested for the presence or absence of, one or more of at least four different nucleic acid variants. An amplification mix and a blocker are added 109 to the partitions. Alternatively, in some embodiments, the amplification mix and blocker(s) may be added to the sample before partitioning. For example, in some cases, the sample may be combined with the amplification mix and blocker(s) prior to partitioning step 105, e.g., such that in step 105 the sample, amplification mix and blocker(s) are partitioned into a plurality of aqueous partitions. Thus, in some cases, step 109 is omitted. The amplification mix may include reagents for digital PCR, such as at least one primer pair, polymerase, dNTPs, any cofactors or ions, and detectably labeled variant-specific probes such as fluorescent hydrolysis probes. Each of the variant-specific probes is specific to one of three of the variants and produces fluorescence of a first color or a second color. For embodiments that include more than three variants and/or more than two channels, those elements will still typically be included. The dPCR will be subject to at least one two-channel readout step for at least three of however many variants are to be detected. The blocker typically is a molecule or complex that inhibits fluorescence from amplification of a fourth one of the variants. That reaction mixture is amplified 113 by subjecting the partitions to conditions that promote amplification. A readout operation is performed to detect 119 fluorescence in at least two channels. In fact, the method 101 may be used with a series of N channel detections 119 to detect for >N variants. The method 101 includes reporting 125 the presence or absence of the three of the variants in the sample based on amounts of the first color and the second color detected from the partitions.

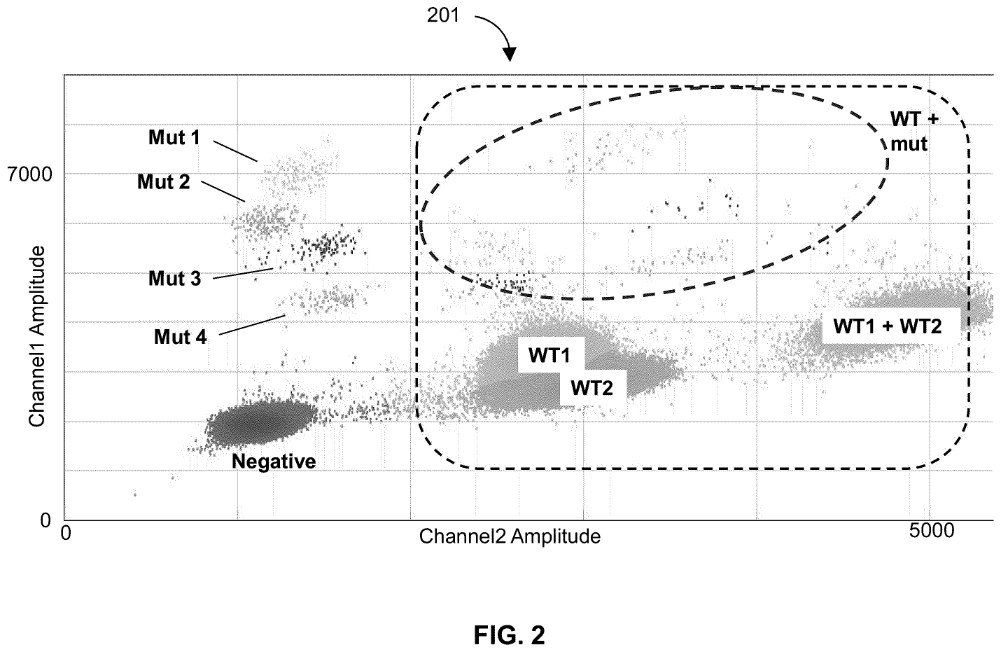

FIG. 2 is a plot 201 that illustrates the multiplex detection of four variants (labeled as Mut 1, Mut 2, Mut 3, and Mut 4) as well as two wild-type alleles (labeled as WT1 and WT2) by a two-channel readout operation from a droplet digital PCR assay, here depicted without the use of a blocker. For the readout shown, the reaction mixture included a hydrolysis probe for Mut 1 with fluorophore 1 (e.g., FAM) read in Channel 1, as well as such a probe for Mut 2. For Mut 3 and Mut 4, probes may be included that are labeled with FAM as well as a small number of probes labeled with fluorophore 2 (e.g., HEX) that is read in Channel 2. Each of WT1 and WT2 are read using hydrolysis probes labeled with fluorophore 2.

For the depicted embodiment, Mut 1, Mut 2, Mut 3, Mut 4, WT1, and WT2 were present in a sample that was partitioned into aqueous droplets along with an amplification mix. Those droplets may be formed by any suitable mechanism, instrument, or technique. For example, those reagents may be loaded into a droplet-digital PCR system such as the system sold under the trademark QX600 by Bio-Rad laboratories. The system may flow the aqueous sample at a preferred dilution through a microchannel to a junction where an aqueous fluid containing the amplification mix is added, downstream of which the aqueous mixture meets a cross flow of an immiscible carrier fluid such as a fluorinated oil. The aqueous reaction mixture breaks off into monodisperse water-in-oil droplets at the junction under co-flow conditions with the oil. Due to the preferred dilution, each droplet will contain zero or a small number of template molecules of nucleic acid from the sample. For example, zero, one, two, or three molecules per droplet may be common. The dilution can be calculated from a reading of nucleic acid quantity in the sample (e.g., optical density) and average fragment length (which may be a known result from sample processing or determined by, e.g., a gel). A surfactant (such a fluorosurfactant) may be added to promote droplet stability. Those aqueous droplets flow down a channel, surrounded by the oil, and may be collected in a suitable vessel such as a well of a 96-well plate. On the droplet-digital PCR system, the plate may be subject to thermocycling such that the approximately tens of thousands of droplets in the well experience thermocycling conditions. Template molecules in the droplets are amplified by means of the primers, polymerase, and co-factors. During amplification, hydrolysis probes anneal to their targets (or amplicons thereof) when those targets are present and the probes are hydrolyzed by the polymerase, releasing fluorophores into the droplets.

Each of the variant-specific probes (e.g., fluorescent hydrolysis probes) is specific to one variant and produces fluorescence of a first color or a second color, read in Channel 1 or Channel 2, respectively. Preferably, at least some of the targets are targeted by a mixture of probes with two or more colors of fluorescent reporter. To illustrate, a first target (Mut 2) may be targeted using only probes with a carboxyfluorescein reporter (FAM), a third target (WT2) may be using only probes with a hexachlorofluorescein reporter (HEX), and a second target (Mut 4) may be targeted with a mixture or probes, some with FAM, some with HEX. More complex multiplexing schemes using more complex probe mixtures are described herein.

After amplification, the digital PCR system can load the droplets-spaced apart by oil-into a readout channel and flow the droplets past a detector. Some embodiments of the system read two optical channels (Channel1 and Channel2) during one readout operation. For example, channels 1 and 2 may read FAM and HEX, or other suitable dyes. Any fluorescence in either channel from each partition is plotted, forming a dot on the plot 201. Interesting phenomena are illustrated by plot 201. All targets can be distinguished in the multiplex assay. However, clusters for droplets that include only WT1, WT2, or WT1 & WT2 occupy much of the space allocated along the Channel 2 axis. To improve discernment of the variants from each other, the invention introduces a blocker.

As used herein, the blocker is a molecule or complex that suppresses any signal from some selected species that is expected to be present in a sample. The selected species to be blocked may be what is anticipated to be a dominant (most numerous) nucleic acid in a sample that also includes minor numbers (less numerous) of other nucleic acids (e.g., variants) of-interest. Viewing the plot 201, it can be seen that blocking any fluorescence from WT1, WT2, or WT1 & WT2 will improve resolution among the variants. The field (oval) of points labeled “WT +mut” in plot 201 will not produce the same signal if a blocker is used, so each point in that field will not have significant contribution from either wild type. That is, any droplet that contains one molecule of a mutant and one molecule of wild type will only produce substantial fluorescence from the mutant. However, remembering that some of the variants (mutants) are detected using a mixture of probes, some of which are detected in Channel 1 and some in Channel 2, it will be appreciated that those mutants are still displaced in a positive direction along the Channel 2 axis in plot 201. Moreover, it would be misleading to think that, with signal from the wild type removed, distinction or resolution is improved by simply re-scaling the Channel 2 axis. In fact, the blocker decreases a quantity of free fluorophore contributed by wild type in certain partitions. The total input excitation energy of the instrument may be left unchanged (for example, the stimulus LED may be operated at constant energy) but the relative proportions of free fluorophores available to absorb those photons has been changed by the blockers. Thus, partitions with two mutant molecules (or three, four, etc.) will absorb the same quantity of excitation energy. Partitions that include a mixture of mutant and wild type molecules will absorb different amounts of energy with the wild type blocked depending on whether the mutant is targeted by zero, one, or two probes specific to Channel 2. Remembering that some variants are targeted only by probes that give signal in Channel 1, some by probes for Channel 2, and some by a mixture of Channel 1 and Channel 2 probes, and remembering that each partitions may include 0, 1, 2, 3, 4, or more molecules, blocking the wild-type but keeping the excitation energy constant, the plot 201 of droplets will show a greater spread over the Channel 2 axis (with the wild-type blocked) as the variant specific probes use a greater proportion of the excitation energy.

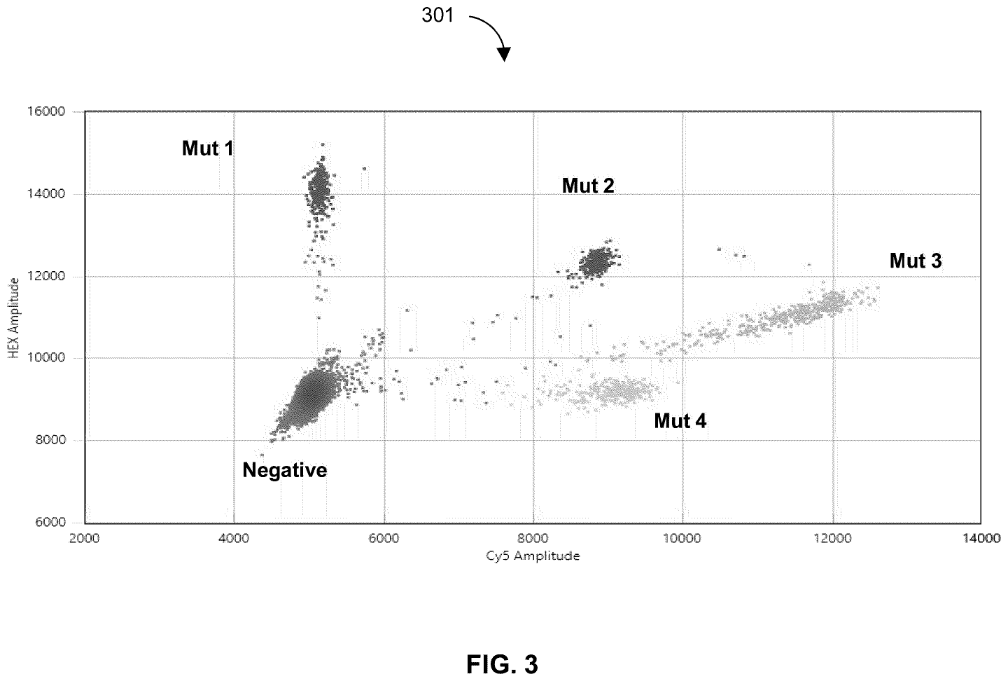

FIG. 3 shows a plot 301 that is obtained when using a blocker to block WT1 and WT2 while probing for Mut 1, Mut 2, Mut 3, and Mut 4 using a set of fluorescent probes that have four target-specific oligonucleotides and two fluorophores. As drawn, the plot 301 was generated using an oligonucleotide that hybridizes specifically to Mut 1 (oligo 1), an oligonucleotide that hybridizes specifically to Mut 2 (oligo 2), an oligonucleotide that hybridizes specifically to Mut 3 (oligo 3), and an oligonucleotide that hybridizes specifically to Mut 4 (oligo 4). The probe set includes only two fluorophores, hexachlorofluorescein (HEX), cyanine5 (CY5). To generate the plot 301, the probe set may be used that includes six probes, as follows:
oligo 1-HEX,
oligo 2-HEX,
oligo 2-Cy5,
oligo 3-HEX,
oligo 3-Cy5, and
oligo 4-Cy5.

To achieve the cluster separation in plot 301, Mut 2 was probed with majority oligo 2-HEX probe and minor amount of the oligo 2-Cy5 probe, while Mut 3 was probed with a minor amount of the oligo 3-HEX probe with the majority being the oligo 3-Cy5 probe. The results shown on plot 301 illustrate radial multiplexing in dPCR with a blocker. Due to the blocker, all of the variants in the sample are shown as clusters that can be distinguished from one another. In fact, cluster detection can be performed in software, and the variant makeup of the sample can be called by software the models Poisson distribution of variants into partitions during dilution and partitioning to identify the concentration of variants in a sample most likely to give the observed clusters.

Note that the cluster labeled “negative” represents partitions for which HEX and Cy5 intensity readings are zero as relevant to the assay. Plot 201 and plot 301 both illustrate approaches to digital multiplexing, here referring generally to the detection of multiple different targets in a single digital assay readout or operation. Digital generally refers to detection methods whereby any given target is detected as present or absent. The polymerase chain reaction (PCR) has been used in digital assays that fall under the description of digital PCR, or dPCR. Digital PCR (dPCR) provides precise, highly sensitive quantification of nucleic acids. Traditional PCR is an end-point analysis that is semi-quantitative because the amplified product is detected by agarose gel electrophoresis. Real-time PCR (or qPCR) uses fluorescence-based detection to allow the measurement of accumulated amplified product as the reaction progresses. qPCR requires normalization to controls (either to a reference or to a standard curve), allowing only relative quantification. Furthermore, variations in amplification efficiency may affect qPCR results. Digital PCR builds on traditional PCR amplification and fluorescent-probe-based detection methods to provide highly sensitive absolute quantification of nucleic acids without the need for standard curves. In some droplet digital PCR Systems (ddPCR), a PCR sample is partitioned into 20,000 droplets. After amplification, droplets containing target sequence are detected by fluorescence and scored as positive, and droplets without fluorescence are scored as negative. Poisson statistical analysis of the numbers of positive and negative droplets yields absolute quantitation of the target sequence.

The concept of digital PCR was described in 1992 by Sykes et al., who recognized that the combination of limiting dilution, end-point PCR, and Poisson statistics could yield an absolute measure of nucleic acid concentration. See Sykes, 1992, Quantitation of targets for PCR by use of limiting dilution, Biotechniques 13:444-449, incorporated by reference. A method was developed whereby a sample is diluted and partitioned to the extent that single template molecules can be amplified individually, each in a separate partition, and the products detected using fluorescent probes. See Vogelstein, 1999, Digital PCR, PNAS 96:9236-9241, incorporated by reference.

Digital PCR improves upon the sensitivity of qPCR and provides for the detection of rare events such as single-nucleotide mutations in a population of wild-type sequences. In conventional qPCR, the signal from wild-type sequences can dominate and obscure the signal from the rare sequence. By minimizing the effects of competition between targets, digital PCR overcomes the difficulties inherent to amplifying rare sequences and allows for sensitive and precise absolute quantification of nucleic acids. Preferred embodiments of digital PCR involve sample partitioning—the division of a sample into discrete subunits prior to amplification by PCR. The sample is prepared in a manner similar to that for real-time PCR but is then separated into e.g., thousands of partitions, each ideally containing either zero or one (or, at most, a few) template molecules. Each partition behaves as an individual PCR reaction and, as with real-time PCR, fluorescent probes are used to identify amplified target DNA. Each partition can then be readily analyzed after amplification to determine whether or not it contains the target sequence. Samples containing amplified product are considered positive (1, fluorescent), and those without product, and thus with little or no fluorescence, are negative (0). The ratio of positives to negatives in each sample is the basis of quantification.

Methods preferably include diluting the sample so that each partitions receives a limited number of target molecules, such as zero, one, two, sometimes three, and a very small number of four or more. The dilution can be calculated so that a majority of partitions receive a target number (e.g., zero or one) of targets. Each target molecule will serve as a template for generation of amplicons in the presence of fluorescence probes.

Partitioning can be accomplished by any suitable mechanism and any suitable type of aqueous partition may be used for digital PCR. Exemplary suitable partitions include droplets, wells in a plate, or other fluid portioning structures.

For example, the partitions may be wells, cavities, pockets, or openings in a pico-, nano-, or microtiter plate or substrate, or fluidic harbors (see, e.g., US 2010/0041046, incorporated by reference). The partitions may be well in a multi-well plate such as a 96-well plate, 384 well plate, a 1536 well plate, a 3456 well plate, or a 9600 well plate. The partitions may be separate chambers (see, e.g., US 20210178395, incorporated by reference). The partitions may be distinct regions defined within a fluidic device (see, e.g., US 20200269248, incorporated by reference). In certain embodiments, the partitions are a plurality of droplets that are formed simultaneously by mixing together and vortexing an aqueous fluid and oil. In preferred embodiments, the partitions are droplets of an emulsion such as a water-in-oil (W/O) emulsion or a water-in-oil-in-water (W/O/W) emulsion.

Preferred embodiments use aqueous partitions in an immiscible liquid, e.g., slugs or droplets surrounded or separated by immiscible carrier fluid such as an oil within a microfluidic device. Aqueous droplets in an immiscible carrier liquid may be formed by microfluidic handling. A microfluidic device may use channels to mix samples and reagents and form droplets in an immiscible carrier fluid. Droplets may be formed within a partitioning section or subunit of a digital PCR instrument or system that uses, e.g., channels to flow an aqueous mixture into an intersecting stream of carrier oil.

The unique sample partitioning step of digital PCR, paired with Poisson statistical data analysis, allows higher precision than traditional PCR and qPCR methods. Accordingly, digital PCR is particularly well suited for applications that require the detection of small amounts of input nucleic acid or finer resolution of target amounts among samples, for example, rare sequence detection, copy number variation (CNV) analysis, and gene expression analysis of the rare targets.

Techniques used in digital PCR include PCR amplification on a microfluidic chip. See Ottesen, 2006, Microfluidic digital PCR enables multigene analysis of individual environmental bacteria, Science 314:1464-1467, incorporated by reference. Other systems involve separation onto microarrays (Morrison, 2006, Nanoliter high-throughput quantitative PCR, Nucleic Acids Res 34:e123, incorporated by reference) or spinning microfluidic discs (Sundberg, 2010, Spinning disk platform for microfluidic digital polymerase chain reaction, Anal Chem 82:1546-1550, incorporated by reference) and droplet techniques based on oil-water emulsions (Hindson, 2011, High-throughput droplet digital PCR system for absolute quantitation of DNA copy number, Anal Chem 83:8604-8610, incorporated by reference), as in droplet digital PCR (ddPCR) systems such as the ddPCR system sold under the trademark QX200 by Bio-Rad Laboratories, Inc. (Hercules, CA).

In such digital assays, each reaction mixture (e.g., partition) is interrogated for the presence or absence of target. The detection of target may be performed using optical density, intercalating dyes, ethidium bromide, change in pH, release of pyrophosphate, or any other suitable method for detecting a target.

Some embodiments discussed herein use fluorescent hydrolysis probes such as the probes sold under the trademark TAQMAN by Thermo Fisher Scientific (Waltham, MA). These probes generate a fluorescent signal when their complementary target is amplified by PCR in the presence of the probe. Such probes include an oligonucleotide backbone that anneals in a sequence specific manner to a target of that probe, plus a fluorophore and a quencher. Methods may include in some embodiments thermocycling the droplets within a reaction tube or well of a plate. During amplification with polymerase, exonuclease activity digests the oligonucleotide backbone of any bound probe, separating the fluorophore from the quencher, allowing the fluorophore to fluoresce during a readout step. In some specific embodiments, to read fluorescence, methods may include flowing the droplets (e.g., one-at-a-time) past a detector (and optionally a light source for excitation of fluorophores).

Alternatively, the probes include molecular beacon probes that anneal to a sequence of amplicons, that may or may not be tagged. Tags may occur on the ends of the amplicons or in the middle of the amplicon sequence. Molecular beacon probes may include an oligonucleotide loop backbone that anneals in a sequence specific manner to a substrate of that probe, complementary stem sequences, plus a fluorophore and a quencher. During the amplification reaction, the complement to the loop sequence is synthesized followed by hybridization of the loop sequence, separating the fluorophore and quencher, allowing the fluorophore to fluoresce during the readout step.

Fluorescent probes may include one or more quenchers and one or more fluorophores, in one or more colors.

In other embodiments, other fluorescent probe systems known in the art can be used, for example, any probe system described in Storts DR. Alternative probe-based detection systems in quantitative PCR. J Mol Diagn. 2014 November; 16(6):612-4, which is herein incorporated by reference.

For either fluorescent hydrolysis probes or molecular beacon probes, the probe may bind to a target sequence between two primers or may bind to the tails of one or both primers used in the amplification process. While the description of the invention refers to fluorescent hydrolysis probes, the invention is equally operable by other probe types which also provide a fluorescent signal (e.g., molecular beacon probes, FRET-based probes, Scorpions® probes, etc.). To read fluorescence, methods may include flowing the droplets (e.g., one at a time) past one or more detector (and optionally a light source for excitation of fluorophores). Alternatively, to read fluorescence, methods may include using immobilized partitions, including but not limited to droplets, within a 2 or 3D array and imaging with one or more detectors (and optionally a light source for excitation of fluorophores). Alternatively, to read fluorescence, methods may include using immobilized partitions, including but not limited to physical partitions, such as wells, for example in a 2D array, and imaging with one or more detectors (and optionally a light source for excitation of fluorophores).

In another embodiment, intercalating dyes, such as SYBR green, can be used in combination with one or more fluorescent probes during the amplification process.

Some dPCR systems read two or more channels (or two or more colors of fluorescence) together at a time. For example, some ddPCR system flow droplets past two color detectors and, for each droplet, two colors are read, simultaneously. Some such platforms have multiple, e.g., six, color channels, but typically read two colors at a time from each partition to generate results such as plot 201 and plot 301. Alternatively, some dPCR systems read two or more channels sequentially.

Plot 201 and plot 301 both illustrate digital multiplexing, which can be seen in those plots because a number of different targets are each independently detected from the two-color readings. One difference between plot 301 and plot 201 is that a selection step has been added to the digital multiplexing assay. In the depicted embodiments, a blocker (e.g., against wild type) was used in generating plot 301 but was not used in generating plot 201. That blocker, by suppressing signal from wild-type molecules, is selective against a predominant molecule present in the sample and selective for molecules present in the sample as minor fractions of the potential targets. Thus, plot 301 illustrates selective digital multiplexing. To generate selective digital multiplexing results illustrated in plot 301, a certain selection of reagents (probes and blocker) may be used.

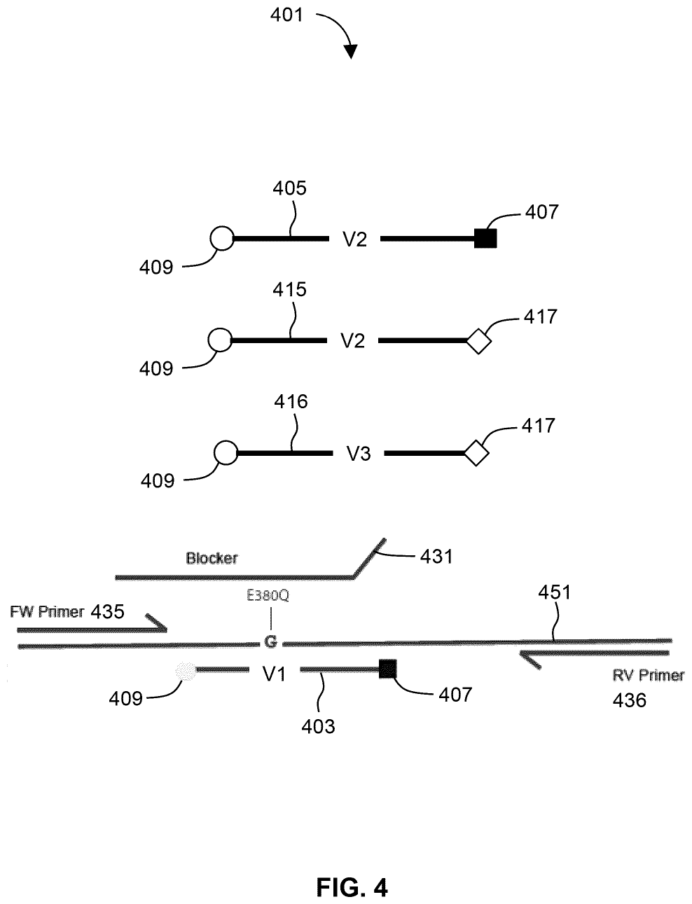

FIG. 4 illustrates reagents 401 including probes and blocker used in certain embodiments of selective digital multiplexing. In the illustrated embodiment, a sample includes (or potentially includes, or is suspected of including, or is being tested for the presence or absence of) fragments of a wild-type gene and up to three variants (e.g., 0, 1, 2 or all 3), V1, V2, and V3. The variant V1 is drawn as molecule 451 while the wild type, V2, and V3 molecules are not shown. The reagents 401 used for selective digital multiplexing include a blocker 431, a V1-dye1 probe 403, a V2-dye1 probe 405, a V2-dye2 probe 415, and a V3-dye 2 probe 416. All probes as shown have a quencher 409. Some of the probes have a first fluorophore 407 and some of the probes have a second fluorophore 417. The reagents 401 include forward primer 435 and reverse primer 436 as well as other PCR reagents that do not need to be drawn because they will be readily understood by one of skill in the art (e.g., polymerase, co-factors, dNTPs).

When the three variants are amplified in partitions, the probes will release free fluorophore 407, 417. Each partition that included any variant will absorb fluorescence excitation light, which may be provided by a light source such as an LED within a dPCR system. Such a system typically includes at least two channels, sometimes referred to as a color channel, that includes a detector such as a photodiode to detect and record optical signal from each partition. As each partition is read (e.g., as a droplet flows past the photodiodes of two channels) the amplitude of fluorescence from that partition is recorded. The recorded amplitude of the two colors of fluorescence (e.g., HEX and Cy5) may then be plotted. Each partition will provide one of the points plot 301. Note that because the blocker 431 is used, the different variants resolve into well-separated clusters on the plot. In fact, from the two (or more) detection channels of a dPCR system, there are software packages available that create 2D plots showing the clusters and even detect and discriminate the clusters and implement a model based on Poisson statistics to provide quantities of the variants in the sample based on the observed data. One such software packages is the dPCR analysis software sold under the trademark QUANTASOFT by Bio-Rad Laboratories, Inc. Other approaches to “calling” a sample (reporting the presence or absence of variants for which probes were introduced) include the use of available software packages such the software package named “dPCR Cluster Predictor” (dPCP), an R package and a Shiny app for automated analysis of up to 4-plex dPCR data. dPCP can analyze and visualize data generated by multiple dPCR systems carrying out accurate and fast clustering not influenced by the amount and integrity of input of nucleic acids. See De Falco, 2023, Digital PCR cluster predictor: a universal R-package and shiny app for the automated analysis of multiplex digital PCR data, Bioinformatics 39(5):btad282, incorporated by reference.

As discussed, and shown in connection with plot 301, methods of the invention may be used for selective digital multiplex detection of multiple targets with two-channel readout at a time. For example, five targets may be read in two color channels. Some systems and platforms may use more than two color channels. In some embodiments, four or six or more color channels are used, two at a time during readout. For example, the method may include reading the sample for at least seven variants using at least six colors, wherein the detecting step reads two of the six colors, in two channels, at a time.

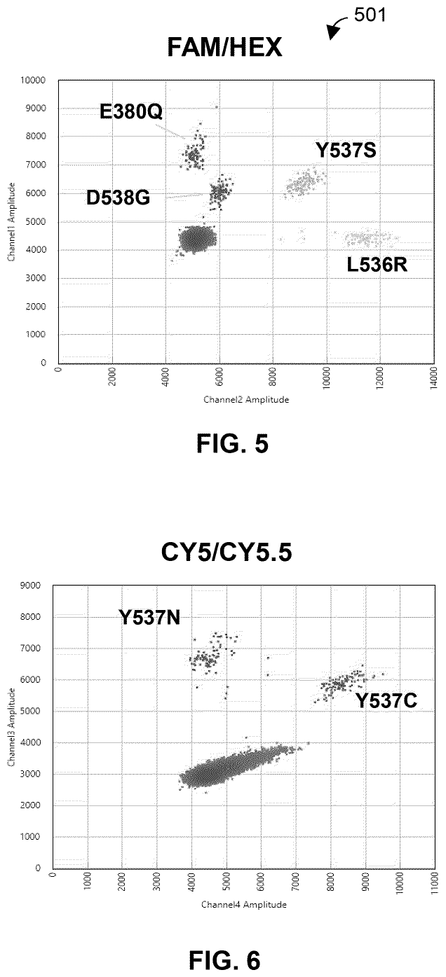

FIG. 5 through FIG. 7 illustrate the use of six color channels to perform selective digital multiplexing for the detection of 8 variants. A selective digital multiplexing assay is performed to detect variants of estrogen receptor 1 that encode the following amino acid substitutions: E380Q, Y537S, D538G, L536R, Y537N, Y537C, S463P, and an internal control. Gene fragments in the sample are 99% wild type and 1% of the fragment are one of the variants.

As shown, the gene is estrogen receptor 1 (ESR1) and the probes are each specific to at least one of: c1138G>C, c.1387T>C, c1607T>G, c.1609T>A, c1610A>C, c1610A>G, and c.1613A>G in the coding sequence of the ESR1 gene.

The sample and reagent are partitioned into aqueous partitions. The reagents include a primer pair for the gene, a blocker for wild-type, mixtures of probes for each variant, and PCR reagents. A dPCR system is used to partition, amplify, read, and analyze the sample. The aqueous sample is partitioned by co-flow into a fluorinated oil to form about 20,000 nanoliter-scale droplets that are then held in one well of a 96-well plate. The ddPCR system thermocycles the plate and transfers the droplets to microfluidic channels that flow the droplets, one-at-a-time, past two photodiodes. The first reading detects fluorescence from carboxyfluorescein (FAM) and hexachlorofluorescein (HEX) in each droplet. A software package makes a 2D plot of the fluorescent readings.

FIG. 5 shows a 2D plot 501 of the reading of FAM and HEX in each droplet.

The second reading detects fluorescence from cyanine5 (CY5) and cyanine5.5 (CY5.5) in each droplet.

FIG. 6 shows a 2D plot of the reading of CY5 and CY5.5 in each droplet.

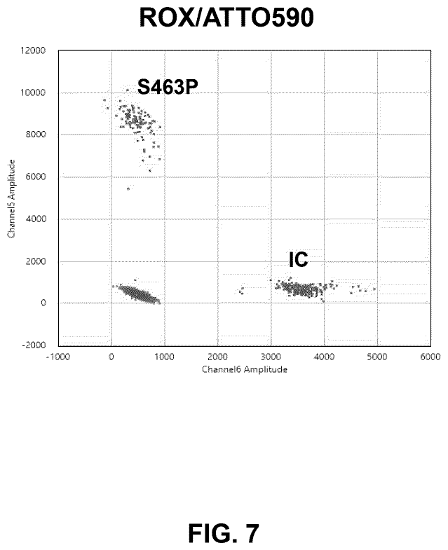

FIG. 7 shows a 2D plot from the final reading, of 5-carboxy-X-rhodamine (5-ROX) and a fluorescent oligonucleotide dye with 594 nm adsorption (ATTO590), from each droplet. In the depicted example, where the targets of-interest are variants, or mutants, of estrogen receptor 1 the blocker suppresses amplification-generated fluorescence of the wild-type, allowing the dPCR readout to have greater discriminating power for the variants, which may each be a very minor fraction (here, 1%) of original sample, relative to the wild type. As shown, the selective digital multiplexing method (selective by virtue of the blocker, digital by virtue of the isolation of template into partitions), includes reading the sample for at least seven variants using at least six colors, wherein the detecting step reads two of the six colors, in two channels, at a time.

Preferably, each variant is targeted with a respective probe or probe combination that includes a sequence specific to that variant and zero or a characteristic amount of the first color or the second color. Using only plot 501 to illustrate, two channels (FAM & HEX) were used to read variants that encode E380Q, Y537S, D538G, and L536R. Each of those four variants were detected with a probe or probe combination.

Referring to E380Q as the first variant, the probes for that variant all included an oligonucleotide specific to that variant and a FAM fluorophore. With D538G as the second variant, the probe combination for that variant include oligonucleotides specific to that variant with about 25% of them linked to HEX and about 75% linked to FAM. For Y537S, about 25% of the probes had FAM and about 75% have HEX. For L536R, all probes are linked to HEX. The exact percentages are not as important as the principle being illustrated: multiple variants can have respective probes each carrying one of two fluorophores with the percentage of a probe having which fluorophore varying progressively over the targets. From such a probe combination, amounts of the first color and the second color detected from the partitions are plotted against respective first and second axes on a plot 501. The variants each form a respective distinct cluster on the plot 501. Notably, the distinct clusters could be separated by radii extending from one point (in the dark, unlabeled cluster corresponding to double-negative droplets). Note that the blocker converts wild type droplets to double-negatives. Because the clusters could be indicated by distinct radii, such a readout could conveniently be referred to as radial multiplexing. Due to the blocker, the assay is selective for the mutations (aka variants). As implemented to generate the plot 501, the assay is performed using droplet digital PCR. Thus the results are a selective digital PCR assay with radial multiplexing.

Such a selective multiplexing assay may be used for any suitable purpose in research, medicine, inquiry, diagnostics, or any other field of endeavor. For example, the depicted methods may be used for the analysis of tumor nucleic acids. In some examples, a patient may have or have had a tumor. Methods of the invention may be used to analyze a sample from that patient for tumor DNA. For example, a liquid biopsy sample such as blood draw may be analyzed for circulating tumor DNA (ctDNA) or to capture, and extract nucleic acid from, circulating tumor cells (CTCs). The blocker can suppress readout from the wildtype, thereby giving exquisite sensitivity for the tumor DNA. Analyzing such a sample by selective digital PCR with radial multiplexing, as shown, can give evidence of the presence of the tumor in the patient. This can be used, for example, to quickly and inexpensively detect minimal residual disease after a cancer treatment.

Thus, it can be seen that method of the invention may use radial multiplexing, which can be implemented by reading two colors from each of a plurality of partitions, plotting intensity of the two colors on a 2D plot with axes for intensity of each color, and identifying clusters of points on the plot. Each cluster will typically be found essentially lying along distinct radius extending from a cluster corresponding to double-negative (no significant fluorescence of either color) partitions. In radial multiplexing, clusters corresponding to distinct targets in the sample are distinguishable according to their different radial directions from the double negative cluster. Clusters can further be distinguished based on radial distance. Thus, five or more targets can be distinguished by providing mixtures of probes with varying amounts of the two fluorescent reporters interrogated in the two channels. Other methods of multiplexing such as fluorescence intensity multiplexing are within the scope of the disclosure. For radial multiplexing, the reporting step may include plotting the amounts of the first color and the second color detected from the partitions as points on a graph and identifying clusters of the points on the graph corresponding to the presence of any of the three variants. The quantities of the respective targets in the sample may be determined by a Poisson model of templates into the partitions that would generate the observed pattern of clusters.

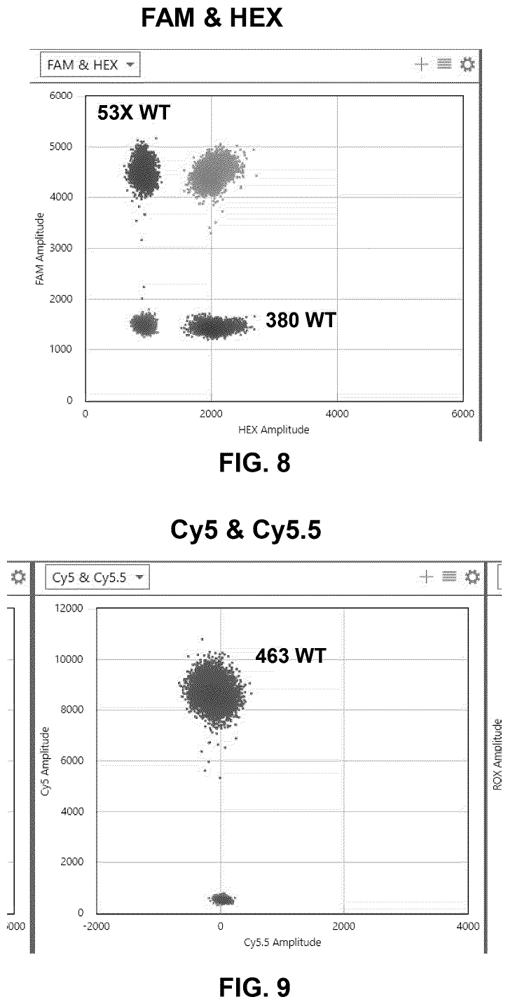

FIG. 8 through FIG. 10 illustrate results of a droplet digital PCR assay for wild type sequences from ESR1 that are homologous to those variants that were interrogated in FIG. 5 through FIG. 7 (an “ESR1 WT assay”). A PCR system with six color channels was used. The system reads two channels at a time and, by virtue of a dPCR software analysis package in a coupled computing system (e.g., QUANTASOFT), produces three plots per instrument run from one well of a 96 well plate in which about 20,000 droplets of sample are held.

FIG. 8 shows reading of WT ESR1 gene with none of the amino acid substitutions at positions 536, 537, 538, or 380, read in FAM and HEX channels.

FIG. 9 shows reading of a WT ESR gene that does not encode a substitution at position 463, read in the Cy5 channel (while the instrument read Cy5 and Cy5.5).

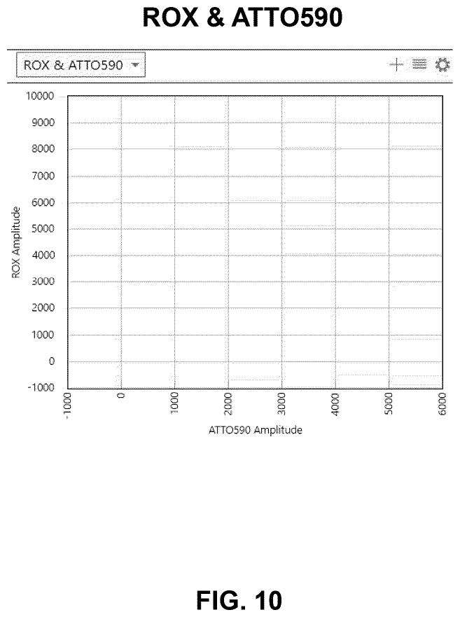

FIG. 10 is a blank plot generated by the instrument in the ESR1 WT assay because the instrument made readings in the ROX and ATTO590 channel, which channels were not used in this assay. The presence of abundant WT nucleic acids shown in the ESR1 WT assay underscores the value of using a blocker in the mutant or variant assay that generated FIG. 5 through FIG. 7. Due to the inclusion of the blocker, true radial multiplexing of all of the cancer-associated variants, can be reliably performed.

Embodiments of the invention are described and illustrated using radial multiplexing. However, other multiplexing techniques are within the scope of the invention. For example, all probes may have a unique color and each variant may be read in its own respective color channel. Other embodiments involve the use of fluorescence intensity multiplexing.

For fluorescence intensity multiplexing, two or three or more different targets can each be given their own detectable label, but the assay can provide those labels in a format that provides a different, distinct fluorescent intensity for each target. For example, a wild type or similar dominant species of a nucleic acid may be blocked with a blocker. Each variant or target of interest may be given its own fluorescent hydrolysis probes. However, each probe may have a unique number of fluorophores (e.g., probes for target 2 may each include an oligonucleotide linked to two fluorophores with only one fluorophore on probes for target 1). Additionally, or alternatively, the probes can be provided in different concentrations. For example, target 1 can be provided with a stoichiometrically restricted quantity of probes calculated such that only about 10% of amplicons of target 1 end up probe-labeled, while target 2 probes may be provided in excess such that 100% of target 2 amplicons are probe labeled. Additionally, or alternatively, amplification efficiency may be controlled between targets such that, for example, target 1 is amplified more efficiently than target 2 and therefore provides greater intensity of fluorescence. This may result from amplicons of different length, different GC content, different annealing temperatures to the probes, unnatural bases and/or different modifications on the backbone of the DNA.

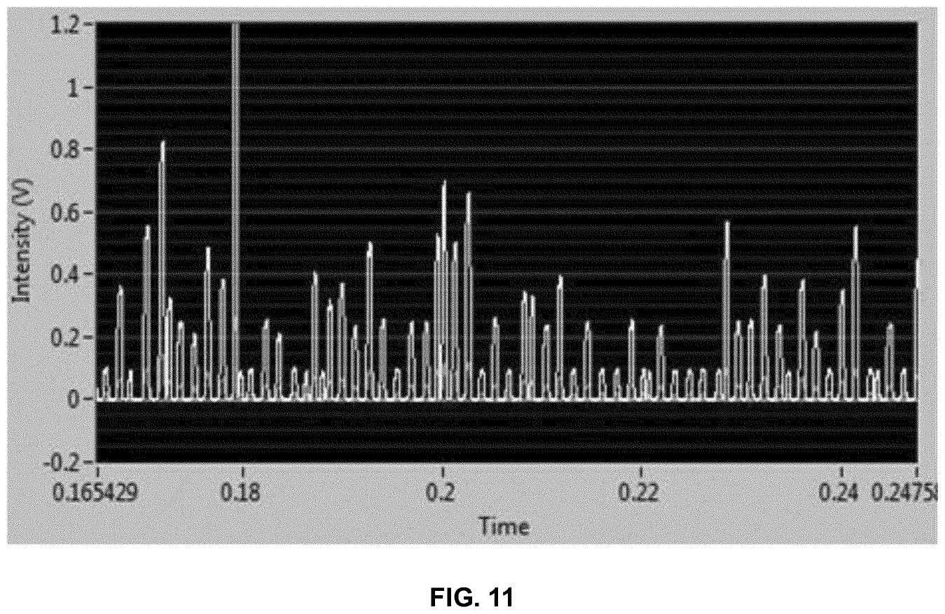

FIG. 11 illustrates results of a fluorescent intensity multiplexing assay. Because only one color channel is read, there is no need for a 2D plot. Partitions that contain wild type largely do not fluoresce due to the blocker. Partitions that contain target 1 fluoresce with an intensity of about 0.23 V due to restricted probe quantities. Partitions that contain target 2 fluoresce at about 0.35 V due to ample probe quantities. Spikes with fluorescence above 0.45 V indicate target 3. Partitions with very high fluorescence reflect multiple templates in one partition due to Poisson distribution in the partitioning. From the fluorescence intensities, template in the original sample can be “called” (calculated, predicted, or reported) by a software package implementing Poisson modeling. As shown the disclosure provides a multiplex digital PCR method that includes dividing a sample that includes, or potentially includes, or is suspected of including, or is being tested for the presence or absence of, one or more of at least four distinct nucleic acid targets into a plurality of aqueous partitions that also include detectably labeled probes for three of the targets and a blocker that inhibits, during amplification conditions, production of a detectable signal from a fourth one of the targets. The method includes exposing the partitions to the amplification conditions and detecting the presence or absence of each of the three of the targets in the sample by reading signal. For radial multiplexing, signal is preferably read in at least two optical channels. The detecting step may be done by plotting signal intensity from each partition on a plot with an axis for each channel, such that each of the three targets, if present in the sample, forms a distinct cluster on the plot. For those targets that are present in the sample, the plot may include clusters that are located along their own radii extending from a point (the double-negative point) on the plot. For fluorescence intensity multiplexing, fluorescence intensity need only be read in once channel. Some embodiments of the invention provide for multiplexing five or more targets in a two-channel dPCR reading, e.g., via radial multiplexing or a single channel reading, e.g., via fluorescence intensity multiplexing.

In highly multiplex embodiments of methods of this disclosure, the sample may include, or potentially include, or is suspected of including, or is being tested for the presence or absence of, one or more of at least eight targets and the method may include detecting the presence or absence of least seven of the eight targets by three two-color reading operations over a total of six colors. The four distinct nucleic acid targets may include homologous genetic sequences. The targets may be variants of a wild-type gene, where the variants may be of, e.g., clinical significance, and the blocker is used to suppress the wild-type out of digital PCR readouts. For example, the blocker may inhibit detection of a wild-type sequence of a gene where each of the targets include a mutated portion of the gene (i.e., that includes a mutation, relative to the wild-type sequence). Exemplary genes may include BRAF, EGFR, KRAS, NRAS, PIK3CA, or ESR1. The sample may include tumor DNA from a subject, the blocker suppresses detection of wild-type sequences from non-tumor DNA from the subject, and detection of the three targets (presence or absence of each) shows the presence or grade of a tumor in the subject.

Features described above as well as those claimed below may be combined in various combinations without departing from the scope of the invention. The following examples illustrate some possible, non-limiting combinations:
(A1) A target detection method comprising partitioning, into a plurality of aqueous partitions, a sample that comprises, or potentially comprises, or is suspected of comprising, or is being tested for the presence or absence of, one or more of at least four different nucleic acid variants. The partitions include amplification reagents, variant-specific probes, wherein each probe is specific to one of three of the variants and produces fluorescence of a first color or a second color, and a blocker that inhibits fluorescence from amplification of a fourth one of the variants. The partitions are subjected to conditions that promote amplification, and the presence or absence of the three of the variants in the sample are reported based on amounts of the first color and the second color detected from the partitions.
(A2) For the method denoted as (A1), the probes comprise fluorescent hydrolysis probes.
(A3) For the method denoted as (A1), the probes comprise molecular beacon probes.
(A4) For the method denoted as any one of (A1) to (A3), the fourth one of the variants is a wild type, the three of the variants are mutant versions of the wild type, and optionally the blocker comprises an oligonucleotide with no fluorescent label.
(A5) For the method denoted as any one of (A1) to (A4), the subjecting step comprises thermocycling the partitions; and optionally the partitions are droplets, and the subjecting step comprises thermocycling the droplets, optionally within a reaction tube or well of a plate, and further optionally wherein the reporting step comprises flowing the droplets one-at-a-time past an excitation source and a detector.
(A6) For the method denoted as any one of (A1) to (A5), the method further comprises detecting in the partitions the presence or absence of at least seven variants using at least six colors, wherein the detecting step reads the at least six colors, in two or more channels, at a time.
(A7) For the method denoted as (A6), the six colors are provided by six fluorescent reporters that include one or more of carboxyfluorescein (FAM), hexachlorofluorescein (HEX), cyanine5 (CY5), cyanine5.5 (CY5.5), 5-carboxy-X-rhodamine (5-ROX), and a fluorescent oligonucleotide dye with 594 nm adsorption (ATTO590).
(A8) For the method denoted as any one of (A1) to (A7), the reporting step comprises plotting the amounts of the first color and the second color detected from the partitions as points on a graph and identifying clusters of the points on the graph corresponding to the presence of any of the three variants.
(A9) For the method denoted as any one of (A1) to (A8), the subjecting step includes thermocycling, wherein the amplification reagents include PCR primers and dNTPs, wherein the variant-specific probes include fluorescent probes that each anneal to a sequence of one of three of the variants, and wherein the blocker comprises an oligonucleotide that anneals to a wild-type version corresponding to one of the at least four variants and inhibits amplification and signal from the wild-type version or amplicons thereof, optionally wherein the blocker comprises an oligonucleotide that anneals the fourth one of the variants and inhibits amplification and signal from the wild-type version or amplicons thereof.
(A10) For the method denoted as any one of (A1) to (A9), the blocker comprises a binding protein that binds to one of the at least four variants and prevents amplification, and optionally the blocker binds to the fourth one of the variants and prevents amplification.
(A11) For the method denoted as any one of (A1) to (A10), the reporting step includes detecting colors from the partitions, two or more color channels at a time, in one or more detection operations.
(A12) For the method denoted as (A11), the reporting step includes reporting the presence or absence of one or more mutations of a gene being present in the sample, optionally wherein the reporting step includes reporting the presence or absence of one or more of at least seven mutations of a gene being present in the sample.
(A13) For the method denoted as (A12), the gene is estrogen receptor 1 (ESR1) and one or more of the seven mutations are selected from the group consisting of c1138G>C, c.1387T>C, c1607T>G, c.1609T>A, c1610A>C, c1610A>G, and c.1613A>G in the coding sequence of the ESR1 gene.
(A14) For the method denoted as any one of (A1) to (A13), the variants are present in tumor DNA in the sample, and the method (i) includes isolating circulating tumor DNA (ctDNA) from the sample, or (ii) isolating circulating tumor cells (CTCs) from the sample and purifying DNA from the CTCs.
(A15) For the method denoted as any one of (A1) to (A14), each of the three of the variants is targeted with one or more variant-specific probe, or a variant-specific probe combination, that includes a sequence specific to that variant and zero or a characteristic amount of the first color or the second color.
(A16) For the method denoted as (A15), a first probe combination specific to a first variant include only probes with a first sequence and the first color, a second probe combination specific to a second variant includes a subset of probes with a second sequence and the first color and a subset of probes with a second sequence and the second color, and a third probe combination specific to a third variant includes only probes with a third sequence and the second color.
(A17) For the method denoted as any one of (A1) to (A16), amounts of the first color and the second color detected from the partitions are plotted against respective first and second axes on a plot, wherein the three of the variants each form a respective distinct cluster on the plot, wherein the distinct clusters can be separated by radii extending from one point.
(A18) For the method denoted as any one of (A1) to (A17), the sample comprises one or more of at least four different nucleic acid variants.
(A19) For the method denoted as any one of (A1) to (A18), the sample potentially comprises one or more of at least four different nucleic acid variants.
(A20) For the method denoted as any one of (A1) to (A19), the sample is suspected of comprising one or more of at least four different nucleic acid variants.
(A21) For the method denoted as any one of (A1) to (A20), the sample is being tested for the presence or absence of one or more of at least four different nucleic acid variants.
(A22) For the method denoted as any one of (A1) to (A21), the sample comprises at least the fourth variant.
(A23) For the method denoted as (A4), the sample comprises at least the fourth variant.
(B1) A multiplex digital PCR method comprising dividing a sample that potentially comprises one or more of at least three distinct nucleic acid targets and a fourth nucleic acid target into a plurality of aqueous partitions that also include detectably labeled probes for the at least three targets and a blocker that inhibits production of a detectable signal from the fourth target during amplification. The partitions are exposed to amplification conditions, and the presence or absence of each of the at least three targets in the sample is detected by reading signal in two or more optical channels.
(B2) For the method denoted as (B1), the detecting step comprises plotting signal intensity from each partition on a plot with an axis for each channel, wherein each of the three of the targets forms a distinct cluster on the plot when that target is present in the sample.
(B3) For the method denoted as any (B2), the plot includes for targets present in the sample, respective clusters, and the clusters are located along respective radii extending from a point on the plot.
(B4) For the method denoted as any one of (B1) to (B3), a first one of the three targets is detected using only probes that include a first oligonucleotide sequence linked to a label that produces a first color, a second one of the three targets is detected using a mixture of probes that include a first probe comprising a second oligonucleotide sequence linked to a label that produces the first color and a second probe comprising the second nucleotide sequence linked to a label that produces a second color, and a third one of the three targets is detected using only probes that include a third oligonucleotide sequence linked to a label that produces the second color.
(B5) For the method denoted as any one of (B1) to (B4), the blocker is an oligonucleotide that binds to the fourth one of the targets, or copies thereof, and optionally wherein the blocker (a) inhibits binding of any of the detectably labeled probes; or (b) inhibits amplification of the fourth one of the targets; or both (a) and (b).
(B6) For the method denoted as (B5), the blocker includes at least one locked nucleic acid.
(B7) For the method denoted as any one of (B1) to (B6), the at least three distinct nucleic acid targets include at least seven targets, and the method further comprises detecting the presence or absence of at the at least seven targets by three, two-color reading operations over a total of six colors.
(B8) For the method denoted as any one of (B1) to (B6), the fourth nucleic acid target is a wild-type sequence of a gene, and each of the at least three targets include a portion of the gene that includes a mutation, relative to the wild-type sequence.
(B9) For the method denoted as (B8), the gene is selected from the group consisting of BRAF, EGFR, KRAS, NRAS, PIK3CA, and ESR1.
(B10) For the method denoted as any one of (B1) to (B9), the sample includes tumor DNA from a subject, the blocker suppresses detection of wild-type sequences from non-tumor DNA from the subject, and wherein detecting the presence or absence of each of the three of the targets shows the presence or grade of a tumor in the subject.
(B11) For the method denoted as any one of (B1) to (B10), the blocker comprises a protein that binds specifically to the fourth one of the targets or amplicons thereof.
(B12) For the method denoted as (B11), the blocker comprises an RNA-guided binding protein.
(B13) For the method denoted as any one of (B1) to (B12), the method further comprises estimating quantities of each of the three of the targets in the sample by modelling, using a computer system, a Poisson distribution of the three of the targets that would give the reading of the signal in the two or more optical channels.
(B14) For the method denoted as any one of (B1) to (B13), the four distinct nucleic acid targets include (i) a first mutation of a wild-type sequence, (ii) a second mutation of the wild-type sequence, (iii) a third mutation of the wild-type sequence, and (iv) the wild-type sequence.
(B15) For the method denoted as (B14), the detectably-labeled probes include: a first probe that includes a first oligonucleotide that anneals to the first mutation with a fluorophore of a first color linked to the first oligonucleotide, a second probe that includes second oligonucleotide that anneals to the second mutation with the fluorophore of the first color linked to the second oligonucleotide, a third probe that includes the second oligonucleotide that anneals to the second mutation with a fluorophore of a second color linked to the second oligonucleotide, and a fourth probe that includes a third oligonucleotide that anneals to the third mutation with a fluorophore of a third color linked to the third oligonucleotide.
(B16) For the method denoted as any one of (B1) to (B15), the method further includes detecting the presence or absence of each of five distinct nucleic acid targets in two optical channels, wherein the five distinct nucleic acid targets include target one, target two, target three, target four, and target five, and wherein the detectably labeled probes include fluorescent hydrolysis probes with five respective oligonucleotides, wherein: all probes for target one have a first fluorophore, a majority of probes for target two have the first fluorophore and a remainder of the probes for target two have a second fluorophore, probes for target three have approximately equal amounts of the first and the second fluorophore, a minority of probes for target four have the first fluorophore and a remainder of the probes for target four have a second fluorophore, and all probes for target five have the second fluorophore.
(B17) For the method denoted as (B16), the five distinct nucleic acid targets are variants of a wild-type gene sequence, and the blocker inhibits a primer from annealing to the wild-type gene sequence or amplicons thereof.
(B18) For the method denoted as any one of (B1) to (B17), the method further includes detecting the presence or absence of each of five distinct nucleic acid targets in the two optical channels, wherein the five distinct nucleic acid targets include target one, target two, target three, target four, and target five, and wherein the detectably labeled probes include fluorescent probes with five respective oligonucleotides, each linked to one of a first and second fluorophore such that no two of the five distinct nucleic acid targets are probed with matching quantities of the first and second fluorophore.
(B19) For the method denoted as any one of (B1) to (B18), the four distinct nucleic acid targets include homologous genetic sequences.
(B20) For the method denoted as any one of (B1) to (B19), the sample comprises one or more of at least four different nucleic acid variants.
(B21) For the method denoted as any one of (B1) to (B20), the sample potentially comprises one or more of at least four different nucleic acid variants.
(B22) For the method denoted as any one of (B1) to (B21), the sample is suspected of comprising one or more of at least four different nucleic acid variants.
(B23) For the method denoted as any one of (B1) to (B22), the sample is being tested for the presence or absence of one or more of at least four different nucleic acid variants.
(B24) For the method denoted as any one of (B1) to (B23), the sample comprises at least the fourth variant.
(C1) A target detection method comprising providing a sample comprising a wild-type sequence and optionally one or more of at least three variant sequences. The sample is partitioned into a plurality of partitions, wherein the partitions further comprise at least three variant-specific probes, each probe being specific to one of the variant sequences and producing fluorescence of a first color or a second color; and a blocker that inhibits fluorescence from amplification of the wild-type sequence. The partitions are subjected to conditions that promote amplification. A level of fluorescence for the first color and the second color are detected from the partitions.
(C21) For the method denoted as (C1), the variant-specific probes comprise fluorescent hydrolysis probes.
(C3) For the method denoted as (C1), the variant-specific probes comprise molecular beacon probes.
(C4) For the method denoted as any one of (C1) to (C3), the blocker comprises an oligonucleotide with no fluorescent label.
(C5) For the method denoted as any one of (C1) to (C4), the subjecting step comprises thermocycling the partitions, and optionally the partitions are droplets, and the subjecting step comprises thermocycling the droplets, optionally within a reaction tube or well of a plate, and further optionally wherein the detecting step comprises flowing the droplets one-at-a-time past an excitation source and a detector.
(C6) For the method denoted as any one of (C1) to (C5), the sample optionally comprises one or more of at least seven variant sequences, the partitions comprise at least seven variant-specific probes fluorescing in one of six colors, wherein the detecting step reads the six colors, in two or more channels, at a time.
(C7) For the method denoted as (C6), the six colors are provided by six fluorescent reporters that include one or more of carboxyfluorescein (FAM), hexachlorofluorescein (HEX), cyanine5 (CY5), cyanine5.5 (CY5.5), 5-carboxy-X-rhodamine (5-ROX), and a fluorescent oligonucleotide dye with 594 nm adsorption (ATTO590).
(C8) For the method denoted as any one of (C1) to (C7), the method further comprises: plotting the amounts of the first color and the second color detected from the partitions as points on a graph, and identifying clusters of the points on the graph corresponding to the presence of any of the three variants.
(C9) For the method denoted as any one of (C1) to (C8), the subjecting step includes thermocycling, wherein the variant-specific probes include fluorescent probes that each anneal to a sequence of one of the three of the variants, and wherein the blocker comprises an oligonucleotide that anneals to a wild-type sequence to prevent variant-specific probes from binding to the wild-type sequence.
(C10) For the method denoted as any one of (C1) to (C9), the blocker comprises a binding protein that binds to the wild-type sequence and prevents amplification.
(C11) For the method denoted as any one of (C1) to (C10), the detecting step detects two or more color channels at a time, in one or more detection operations.
(C12) For the method denoted as (C11), the method comprises reporting the presence or absence of one or more variant sequences of a gene in the sample, optionally wherein the method comprises reporting the presence or absence of at least seven variant gene sequences in the sample.
(C13) For the method denoted as (C12), the gene is estrogen receptor 1 (ESR1) and one or more of the seven variant sequences are mutations selected from the group consisting of c1138G>C, c.1387T>C, c1607T>G, c.1609T>A, c1610A>C, c1610A>G, and c.1613A>G in the coding sequence of the ESR1 gene.
(C14) For the method denoted as any one of (C1) to (C13), the variants are present in tumor DNA in the sample, and the method further comprises: (i) isolating circulating tumor DNA (ctDNA) from the sample; or (ii) isolating circulating tumor cells (CTCs) from the sample, and purifying DNA from the CTCs.
(C15) For the method denoted as any one of (C1) to (C14), each of the three variant sequences is targeted with one or more variant-specific probe that includes a sequence specific to that variant and a characteristic amount of the first color or the second color.
(C16) For the method denoted as (C15), a first probe combination specific to a first variant sequence includes only probes with a first sequence and the first color, a second probe combination specific to a second variant includes a subset of probes with a second sequence and the first color and a subset of probes with a second sequence and the second color, and a third probe combination specific to a third variant includes only probes with a third sequence and the second color.
(C17) For the method denoted as any one of (C1) to (C16), the method further comprises plotting amounts of the first color and the second color detected from the partitions against respective first and second axes on a plot, wherein each variant sequence forms a respective distinct cluster on the plot, and wherein the distinct clusters can be separated by radii extending from a single point.
(C18) For the method denoted as any one of (C1) to (C17), the subjecting step includes thermocycling, wherein the variant-specific probes include fluorescent probes that each anneal to a sequence of one of the three of the variants, and wherein the blocker comprises an oligonucleotide that anneals to a wild-type sequence to inhibit amplification of the wild-type sequence; optionally wherein the blocker blocks binding of a primer thereby inhibiting amplification of the wild type sequence; further optionally wherein the blocker blocks binding of a forward primer or binding of a reverse primer thereby inhibiting amplification of the wild type sequence.
(C19) For the method denoted as (C18), the blocker blocks binding of a forward primer to inhibit amplification of the wild type sequence.
(C20) For the method denoted as any one of (C1) to (C19), the sample comprises one or more of at least four different nucleic acid variants.
(C21) For the method denoted as any one of (C1) to (C20), the sample potentially comprises one or more of at least four different nucleic acid variants.
(C22) For the method denoted as any one of (C1) to (C21), the sample is suspected of comprising one or more of at least four different nucleic acid variants.
(C23) For the method denoted as any one of (C1) to (C22), the sample is being tested for the presence or absence of one or more of at least four different nucleic acid variants.
(C24) For the method denoted as any one of (C1) to (C23), the sample comprises at least the fourth variant.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1-71. (canceled)

72. A multiplex digital PCR method comprising:
dividing a sample that potentially comprises one or more of at least three distinct nucleic acid targets and a fourth nucleic acid target into a plurality of aqueous partitions that also include detectably labeled probes for the at least three targets and a blocker that inhibits production of a detectable signal from the fourth target during amplification;
exposing the partitions to amplification conditions; and
detecting the presence or absence of each of the at least three targets in the sample by reading signal in two or more optical channels.

73. The method of claim 72, wherein
a first one of the three targets is detected using only probes that include a first oligonucleotide sequence linked to a label that produces a first color,
a second one of the three targets is detected using a mixture of probes that include a first probe comprising a second oligonucleotide sequence linked to a label that produces the first color and a second probe comprising the second oligonucleotide sequence linked to a label that produces a second color, and
a third one of the three targets is detected using only probes that include a third oligonucleotide sequence linked to a label that produces the second color.

74. The method of claim 72, wherein the detecting step comprises plotting signal intensity from each partition on a plot with an axis for each channel, wherein each of the three of the targets forms a distinct cluster on the plot when that target is present in the sample.

75. The method of claim 74, wherein the plot includes, for targets present in the sample, respective clusters, and the clusters are located along respective radii extending from a point on the plot.

76. The method of claim 72, wherein the blocker is an oligonucleotide that binds to the fourth target, or copies thereof, and optionally wherein the blocker (a) inhibits binding of any of the detectably labeled probes; or (b) inhibits amplification of the fourth one of the targets; or both (a) and (b).

77. The method of claim 76, wherein the blocker includes at least one locked nucleic acid.

78. The method claim 76, wherein the blocker comprises a protein, and optionally wherein the blocker comprises an RNA-guided binding protein.

79. The method of claim 72, wherein the at least three distinct nucleic acid targets include at least seven targets, and the method further comprises detecting the presence or absence of the at least seven targets by three, two-color reading operations over a total of six colors.

80. The method of claim 72, wherein the sample includes tumor DNA, the blocker suppresses detection of wild-type sequences from non-tumor DNA, and wherein detecting the presence or absence of each of the three of the targets shows the presence or grade of a tumor.

81. The method of claim 72, further comprising estimating quantities of each of the three of the targets in the sample by modelling, using a computer system, a Poisson distribution of the three of the targets that would give the reading of the signal in the two or more optical channels.

82. The method of claim 72, wherein the four distinct nucleic acid targets include (i) a first mutation of a wild-type sequence, (ii) a second mutation of the wild-type sequence, (iii) a third mutation of the wild-type sequence, and (iv) the wild-type sequence.

83. The method of claim 82, wherein the wild-type sequence is of a gene, and each of the at least three targets include a portion of the gene that includes a mutation, relative to the wild-type sequence.

84. The method of claim 83, wherein the gene is selected from the group consisting of BRAF, EGFR, KRAS, NRAS, PIK3CA, and ESR1.

85. The method of claim 72, wherein the probes include:
a first probe that includes a first oligonucleotide that anneals to a first of the at least three distinct nucleic acid targets with a fluorophore of a first color linked to the first oligonucleotide;
a second probe that includes second oligonucleotide that anneals to a second of the at least three distinct nucleic acid targets with the fluorophore of the first color linked to the second oligonucleotide;
a third probe that includes the second oligonucleotide that anneals to the second of the at least three distinct nucleic acid targets with a fluorophore of a second color linked to the second oligonucleotide; and
a fourth probe that includes a third oligonucleotide that anneals to a third of the at least three distinct nucleic acid targets with a fluorophore of a third color linked to the third oligonucleotide.

86. The method of claim 72, wherein the method further includes detecting the presence or absence of each of five distinct nucleic acid targets in two optical channels, wherein the five distinct nucleic acid targets include target one, target two, target three, target four, and target five, and wherein the detectably labeled probes include fluorescent hydrolysis probes with five respective oligonucleotides, wherein:
all probes for target one have a first fluorophore,
a majority of probes for target two have the first fluorophore and a remainder of the probes for target two have a second fluorophore,
probes for target three have approximately equal amounts of the first and the second fluorophore,
a minority of probes for target four have the first fluorophore and a remainder of the probes for target four have a second fluorophore, and
all probes for target five have the second fluorophore.

87. The method of claim 86, wherein the five distinct nucleic acid targets are variants of a wild-type gene sequence, and the blocker inhibits a primer from annealing to the wild-type gene sequence or amplicons thereof.

88. The method of claim 72, wherein the method further includes detecting the presence or absence of each of five distinct nucleic acid targets in the two optical channels, wherein the five distinct nucleic acid targets include target one, target two, target three, target four, and target five, and wherein the detectably labeled probes include fluorescent probes with five respective oligonucleotides, each linked to one of a first and second fluorophore such that no two of the five distinct nucleic acid targets are probed with matching quantities of the first and second fluorophore.

89. The method of claim 72, wherein the four distinct nucleic acid targets include homologous genetic sequences.

90. A target detection method comprising:
partitioning, into a plurality of aqueous partitions, a sample that comprises one or more of at least four different nucleic acid variants, wherein the partitions include:
amplification reagents;
variant-specific probes, each probe being specific to one of three of the variants and produces fluorescence of a first color or a second color; and
a blocker that inhibits fluorescence from amplification of a fourth one of the variants;
subjecting the partitions to conditions that promote amplification; and
reporting the presence or absence of the three of the variants in the sample based on amounts of the first color and the second color detected from the partitions.

91. A target detection method comprising:
providing a sample comprising a wild-type sequence and optionally one or more of at least three variant sequences;
partitioning the sample into a plurality of partitions comprising:
at least three variant-specific probes, each probe being specific to one of the variant sequences and producing fluorescence of a first color or a second color; and
a blocker that inhibits fluorescence from amplification of the wild-type sequence;
subjecting the partitions to conditions that promote amplification; and
detecting a level of fluorescence for the first color and the second color from the partitions.
